(19) United States
(12) Patent Application Publication
(10) Pub. No.: US 2008/0269897 A1
(43) Pub. Date: Oct. 30, 2008

(54) IMPLANTABLE DEVICE AND METHODS FOR REPAIRING ARTICULATING JOINTS FOR USING THE SAME

(76) Inventor: ABHIJEET JOSHI, AUSTIN, TX (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(21) Appl. No.: 11/740,561

(22) Filed: Apr. 26, 2007

(51) Int. Cl. A61F 2/44

(52) U.S. Cl. 623/17.11

(57) ABSTRACT

Embodiments of the invention provide an implantable device having a bioresorbable outer layer and a bioactive inner core. The outer layer can be porous, allowing material to pass through and bond with bones. After placement, the outer layer will dissolve into the body over time. The bioactivity, bonding ability, and mechanical properties can be manipulated in many ways. The size, shape, and number of the implantable device may vary, depending upon needs and applications. The implantable device can be introduced into the articulating joint with minimal invasion to act as both a cushion and a load-bearing device, alleviating pain associated with degenerative facet joints, eliminating the need for fusion/fixation procedures, and preserving/restoring the natural function of articulating surfaces.

### FIELD OF THE INVENTION

This invention relates generally to implantable devices. More particularly, the present invention relates to implantable devices useful in reducing pain associated with articulating joints in the human body. Even more particularly, the present invention relates to biocompatible implants which can act as a load bearing device, as a friction reducing cushion between the articulating surfaces and as a separator to minimize the contact between the articulating surfaces, while preserving and/or restoring the natural intended motion.

### BACKGROUND OF THE INVENTION

The facet joints are the articulations or links between the vertebrae in the spine. The facet joints, knees, and elbows are sometimes referred to as synovial joints. A synovial joint allows movement (e.g., bending, twisting, etc.) between two bones. In a synovial joint, the ends of the bones are covered with a material called articular cartilage. This material is a slick spongy material that allows the bones to glide against one another without much friction.

Surrounding the facet joint is a watertight sack made of soft tissue and ligaments. This sack creates what is called the “joint capsule.” Synovium is a membrane that covers all the non-cartilaginous surfaces within the joint capsule. It secretes synovial fluid into the joint, which nourishes and lubricates the articular cartilage. The ligaments are soft tissue structures that hold the two sides of the facet joint together. The ligaments around the facet joint combine with the synovium to form the joint capsule that is filled with the synovial fluid, which decreases the friction. The synovium is separated from the capsule by a layer of cellular tissue that contains blood vessels and nerves.

The nerve fibers can cause pain when they become inflamed and/or dysfunctional due to trauma (injury) or disease(s) (e.g., arthritis). Pain can also be caused by disc degeneration, where the jelly-like substance of the intervertebral disc becomes dry and stiff, losing its cushioning effect and no longer can work as a shock absorber. Disc degenerative disease (DDD) is attributed to the degenerative process in the spine and is a common cause for chronic or recurring back pain. Patients with DDD may have back pain, leg pain, or varying degrees of both. DDD generally leads to loss of disc height and alters the normal spinal biomechanics and motion. Loss of disc height can reduce the separation of opposing facet joints and alter the biomechanics of those joints. The cartilage of the joint may become compromised or destroyed resulting in nerve compression and/or bone-on-bone contact in the joint. Structural instability and nerve compression are causes for persistent and often significant pain. Furthermore, the abnormal movement of the degenerative disc or motion segment forces the facet joints to carry abnormal physiologic loads that, in turn, cause facet degeneration.

Currently, several pain management/treatment options are available. For example, an injection of local anesthetic can temporarily reduce or eliminate the pain. Surgery provides a more permanent solution. For example, pain impulses from the affected facet joints can be blocked by selectively coagulating (e.g., with a radiofrequency wave or a heated wire) the affected sensory nerve fibers. The pain relief from this type of procedure may last from 6 months to 2 years. Other procedures (e.g., spinal fusion, pedicle screw fixation, etch) are more invasive and do not preserve the natural intended motion. Moreover, because the fused/fixed segment does not bend, additional physical stresses are placed upon the adjacent motion segment by the loads of daily body movements. Thus, even with a successful invasive procedure, back pain problems may resurface later.

### SUMMARY OF THE INVENTION

Embodiments of the present invention provide a new solution to alleviate pain associated with articulating joints in the human body. This solution includes an implantable device and a method of placing the implantable device in an articulating joint (e.g., a facet joint). According to the invention, the implantable device is capable of bearing or sharing the load of an articulating joint, reducing friction between the articulating surfaces of the joint, and maintaining separation of the articulating surfaces. Due to the shape and configuration of the implantable device, the delivery would require minimum or negligible invasion.

In embodiments of the invention, the implantable device is made from two distinctly different parts having different features. The first part forms the outer layer of the implantable device and the second part forms the inner core thereof.

In one embodiment, the outer layer is bioresorbable. In one embodiment, the outer layer can be permeable, allowing the fluidic material inside the implantable device to pass through over time. The thickness of the outer layer can vary, depending upon needs and applications. In one embodiment, the bioresorbable material is deformable. In this way, the shape of the implantable device as well as the volume of the fluidic material inside the implantable device can be manipulated where applicable. After the implantable device is placed inside a facet joint, the outer layer will dissolve into the body over time. In one embodiment, the outer layer is permanent and not bioresorbable. According to the invention, the bioresorbability of the outer layer can vary from implementation to implantation, depending upon the load magnitudes and directions experienced by a particular articulating joint.

In one embodiment, the inner core contains a polymeric material which is bioactive. The bioactivity of the polymeric material can be manipulated (e.g., using certain growth factors). The bonding ability of the polymeric material can also be manipulated (e.g., using appropriate bonding agents). Moreover, the mechanical properties of the polymeric material can be suitably optimized (e.g., using different polymeric combinations and reinforcing materials) to preserve and/or restore the natural function of a facet joint. In one embodiment, the polymeric material can be swellable in the body. The swellability may be enhanced by adding a fluid such as saline, when required. The polymeric material can be pre-formed, in-situ curable, or injectable. After the implantable device is placed inside a facet joint, the bioactive polymeric material will bond to the bone over time. In one embodiment, the polymeric material is hydrogel.

As one skilled in the art can appreciate, the size, shape, and configuration of the implantable device disclosed herein can vary according to needs and applications. For example, one embodiment of the implantable device can be in the form of a thin capsule or tablet.

The space between articulating joints can be temporarily expanded or distracted to allow the insertion of one or more. Implantable devices using standard surgical instruments or a special delivery device. Other methods of delivery are also possible (e.g., by injection). According to one embodiment of the invention, after introduction, the implantable device can be contained, held, or otherwise constrained in the intended space via various temporary and/or permanent means (e.g., bioresorbable and/or biocompatible screws, etc.).

Embodiments of the invention can be used alone or in conjunction with other devices such as total disc replacement, nucleus replacement, or annulus repair devices.

This invention provides a number of advantages, including but not limited to the following. The implantable device can provide a surface to enhance lubrication within an articulating joint (e.g., a facet joint). In one embodiment, the implantable device can alleviate pain associated with degenerated facet joints. The implantable device can be introduced into an articulating joint in a minimally invasive procedure. Moreover, both the outer layer and inner core of the implantable device can be made of relatively inexpensive materials previously used in the human body. The implantable device can solve two different problems. First, the implantable device can act as a cushion between the articulating surfaces by avoiding the contact between the sensory serve fibers. This would facilitate to completely eliminate the debilitating pain resulting from the articulating surfaces. Second, it can act as a load-bearing device, offloading some or all of the physiologic loads from the degenerative joints and eliminating the need for spinal fusion/fixation procedures, thereby advantageously preserving and/or restoring the natural function of articulating surfaces.

Other objects and advantages of the invention will be better appreciated and understood when considered in conjunction with the following description and the accompanying drawings

### BRIEF DESCRIPTION OF THE DRAWINGS

A more complete understanding of the present invention and the advantages thereof may be acquired by referring to the following description, taken in conjunction with the accompanying drawings in which like reference numbers indicate like features and wherein.

FIG. 1 is a graphical representation of a cross-sectional view of an articulating joint in which embodiments of the invention may be practiced;

FIG. 2 is a graphical representation of a cross-sectional view of an implantable device for joint repair, according to one embodiment of the invention;

FIG. 3 is a graphical representation of an implantable device for joint repair, according to another embodiment of the invention;

FIG. 4 is a graphical representation of an implantable device for joint repair, according to yet another embodiment of the invention; and

FIGS. 5-7 are graphical representations of minimally invasive methods of delivering an implantable device for joint repair, according to some embodiments of the invention.

### DETAILED DESCRIPTION OF THE INVENTION

The invention and the various features and advantageous details thereof are explained more fully with reference to the non-limiting embodiments detailed in the following description. Descriptions of well known starting materials, manufacturing techniques, components and equipment are omitted so as not to unnecessarily obscure the invention in detail. Skilled artisans should understand, however, that the detailed description and the specific examples, while disclosing preferred embodiments of the invention, are given by way of illustration only and not by way of limitation. Various substitutions, modifications, and additions within the scope of the underlying inventive concept(s) will become apparent to those skilled in the art after reading this disclosure.

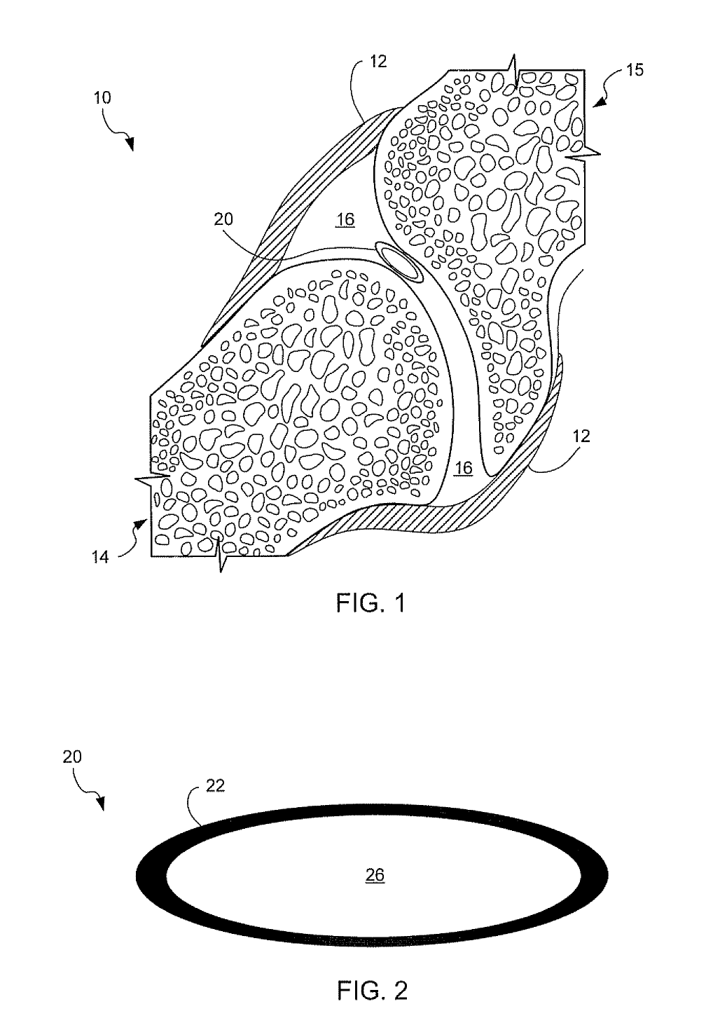

FIG. 1 is a simplified graphical representation of a cross-sectional view of a facet joint 10, which exemplifies an articulating joint. Facet joint 10 includes joint capsule 12 that attaches to bone 14, 15 of an upper and lower vertebra. Joint capsule 12 and bones 14, 15 together define inner cavity 16 that normally holds synovial fluid. Thus, joint capsule 12 surrounds inner cavity 16 on the perimeter, and bones 14, 15 define the upper and lower ends of inner cavity 16. The synovial fluid provides lubrication for the facet joint. If facet joint 10 degenerates, there can be a lessoning of synovial fluid and a reduction in space between bones 14, 15, and painful bone-on-bone contact can occur. Embodiments of the present invention provide an implantable device that can be placed in cavity 16 so that bone-on-bone contact is reduced or eliminated, thereby reducing or eliminating pain for a patient. The implantable device may also provide lubrication for the facet joint and/or share or bear the load thereof.

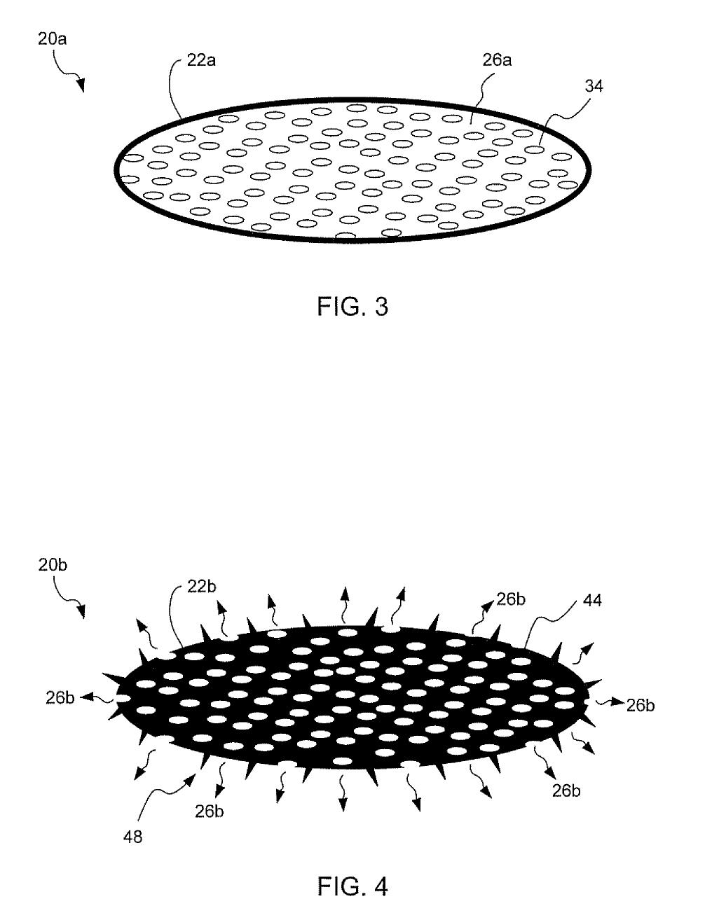

FIG. 2 is a graphical representation of a cross-sectional view of an implantable device 20 useful for facet joint repair, according to one embodiment of the invention. One (or more) of the implantable device(s) 20 can be placed in a facet joint (e.g., the facet joint 10 of FIG. 1) to reduce friction between the articulating surfaces of the facet joint and maintain separation thereof. Due to the shape and configuration of the implantable device 20, the delivery of implantable device 20 can be accomplished with minimal or negligible invasion of the joint.

In one embodiment of the invention, implantable device 20 is made from two distinctly different materials. The first material forms outer layer 22 and the second material forms inner core 26, as shown in FIG. 2.

In one embodiment, outer layer 22 is bioresorbable. In an alternate embodiment, outer layer 22 can be partially bioresorbable or, in some cases, not bioresorbable. The thickness of outer layer 22 can vary and can depend upon the volume of the material inside the implantable device. Outer layer 22 may be made of a bioresorbable material that is also deformable. After implantable device 20 is placed inside a facet joint, outer layer 22 will, according to one embodiment, dissolve into the body over time. This reaction can be manipulated (e.g., by varying the thickness and/or material of outer layer 22) to alter the time for the dissolvance of outer layer 22. By way of example, but not limitation, the material and thickness of outer layer 22 can be selected so that absorption of outer layer 22 will take about between two weeks to three months.

In one embodiment, inner core 26 contains a polymeric material which is bioactive and can bond to the bone (e.g., articulating processes of vertebrae 14, 15 of FIG. 1). The bioactivity of the polymeric material can be manipulated (e.g., using certain growth factors). The bonding ability of the polymeric material can also be manipulated (e.g., using appropriate bonding agents). Moreover, the mechanical properties of the polymeric material can be suitably optimized (e.g., using different polymeric combinations and reinforcing materials) to preserve and/or restore the natural function of a facet joint. In one embodiment, the polymeric material can be swellable in the body. The swellability may be enhanced by adding a fluid such as saline, when required. The polymeric material can be pre-formed, in-situ curable, or injectable. After the implantable device is placed inside a facet joint, the bioactive polymeric material will bond to the bone over time. In one embodiment, the polymeric material is hydrogel.

Implantable device 20 can be in the form of a thin capsule, a tablet or any suitable form, including free form. As one skilled in the art can appreciate, the size, shape, and configuration of implantable device 20 can vary according to needs and applications. Thus, implantable device 20 as illustrated in FIG. 2 is meant to be exemplary and not to be construed as limiting.

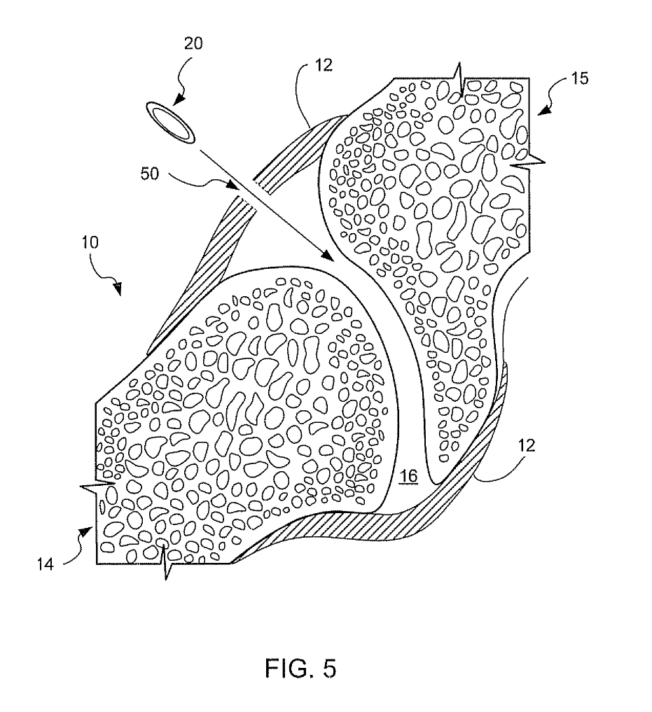

FIG. 3 is a graphical representation of an implantable device 20a useful for facet joint repair, according to another embodiment of the invention. In this example, outer layer 22a is porous. Thus, after implantable device 20a is positioned in place, the bioactive material of inner core 26a can permeate (e.g., through holes 34) and bond with the bones of the facet joint (e.g., bones 14 and 15 of FIG. 1). This reaction can be manipulated to alter the time for bone bonding (e.g., by varying the number, shape, and/or size of holes 34 which determine the porosity, and/or the thickness and/or material of the outer layer 22a). During implantation of device 20a, saline can be added to increase swelling of inner core 26a and hasten bonding of inner core 26a with the bones of the facet joint.

As will be described in detail below, the implantable device disclosed herein can be delivered or otherwise deployed to a desired place/position in an articulating joint (e.g., a facet joint) in many ways. Additionally, the implantable device according to the present invention can be contained or otherwise restricted in the desired position temporarily (bioresorbable) or permanently (bioactive and/or non-bioresorbable). As one of ordinary skill in the art can appreciate, embodiments of the implantable device disclosed herein are all biocompatible. Many temporary arrangements (also referred to as space containers) are possible. For example, one or more bioresorbable screws can be used to constrain the device for an intended period of time. The bioresorbable screw(s) can have at least a similar or longer resorbing time than the outer layer of the device.

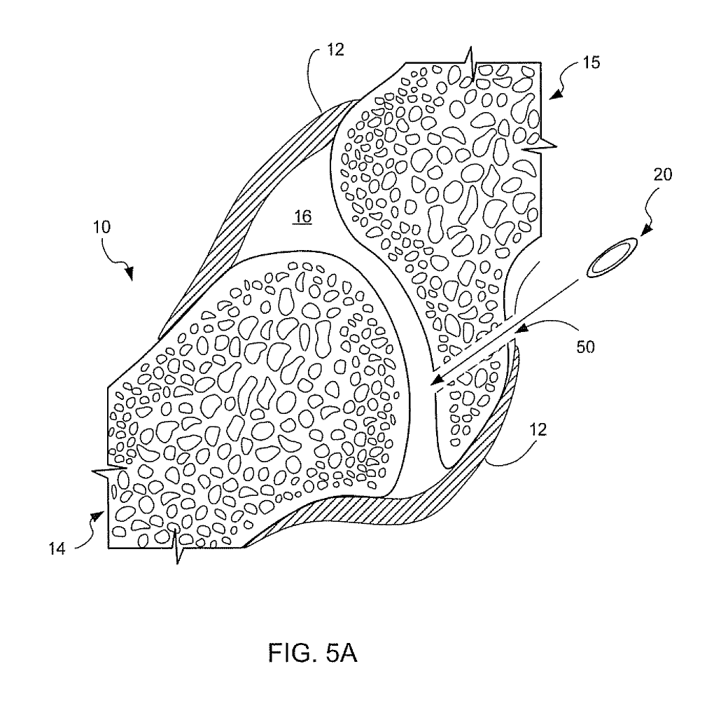

FIG. 4 is a graphical representation of an implantable device 20b useful for facet joint repair, according to yet another embodiment of the invention. In this example, outer layer 22b has a plurality of protruding elements or keels 48 for hooking, anchoring, or otherwise securing implantable device 20b in place after delivery. The plurality of protruding features 48 can be made of the same bioresorbable material as outer layer 22b. The number, shape, and size of protruding features 48 can vary per application (e.g., to achieve a particular length of resorbing time). In one embodiment, outer layer 22b can be permeable (e.g., via holes 44) to allow inner core material 26b to pass through and bond with the bones in the face joint. Additional temporary and/or permanent space container(s) may also be utilized to constrain device 20b in a desired place/position.

For permanent arrangement, any standard biocompatible material can be used to restrict the device in place, even after bone bonding occurs. Such a permanent space container can also be bioactive, which would enhance the bone bonding, yet facilitating a flexible, compliant and stiff enough device, which would serve its intended purpose as described above. Furthermore, embodiments of the implantable device disclosed herein can be used alone or in conjunction with other devices such as total disc replacement, nucleus replacement, or annulus repair devices.

Below describes exemplary methods for placing the implantable device in an articulating joint. The articulating joints have a small gap between the bones (e.g., a 4-6 mm space between bones 14, 15 of FIG. 1) which could be temporarily expanded or distracted to allow the insertion of one embodiment of the implantable device. After it is determined that a facet joint is in need of repair, it should be determined whether the facet joint should be distracted prior to introduction of one or more capsules into the facet joint. If needed (i.e., the size of the cavity is insufficient to allow introduction of a capsule), the distraction of the facet joint can be accomplished through techniques known to those having ordinary skill in the art using standard or customized surgical instruments (e.g., tongue depressors, ramped needles, biocompatible screws, wedges, etc.).

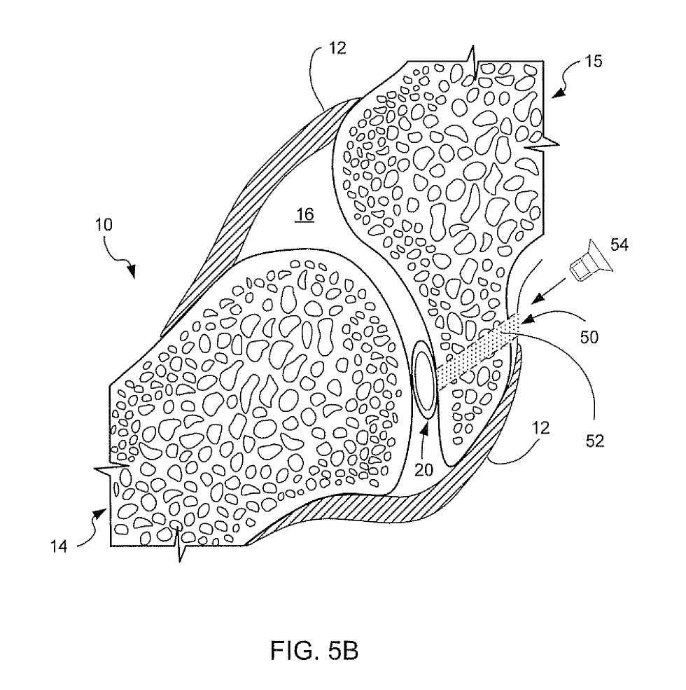

As illustrated in FIG. 5, one embodiment of implantable device 20 can be introduced into cavity 16 of facet joint 10 through hole 50. Depending upon the base material configuration, implantable device 20 can simply be placed in the desired space using tweezers or it can be injected (e.g., using a suitable syringe fitted with a hypodermic needle or cannula). Hole 50 may be created in various ways. For example, FIG. 5 illustrates that hole 50 may be created by an incision in joint capsule 12 and FIG. 5A illustrates that hole 50 may be created by drilling through bone 15. Hole 50, which needs not be circular in shape, is of sufficient size to permit the insertion of implantable device 20 into cavity 16 within facet joint 10. Hole 50 may heal naturally or may be sutured, patched, or filled with a suitable material. For example, after implantable device 20 is introduced into cavity 16, hole 50 (i.e., the drilled conduit in bone 15) can be filled with bone material, adhesive, or other filler 52, then capped or plugged as shown in FIG. 5B. In one embodiment, a metal screw 54 or a screw formed of a material that forms bone over time can be used to close the drilled conduit. In one embodiment, implantable device 20 can be inserted into a temporary space container such as a balloon (not shown) that has been previously placed in facet joint 10. The balloon, or some other containment system, serves to temporarily constrain the implantable device 20 as it is being delivered.

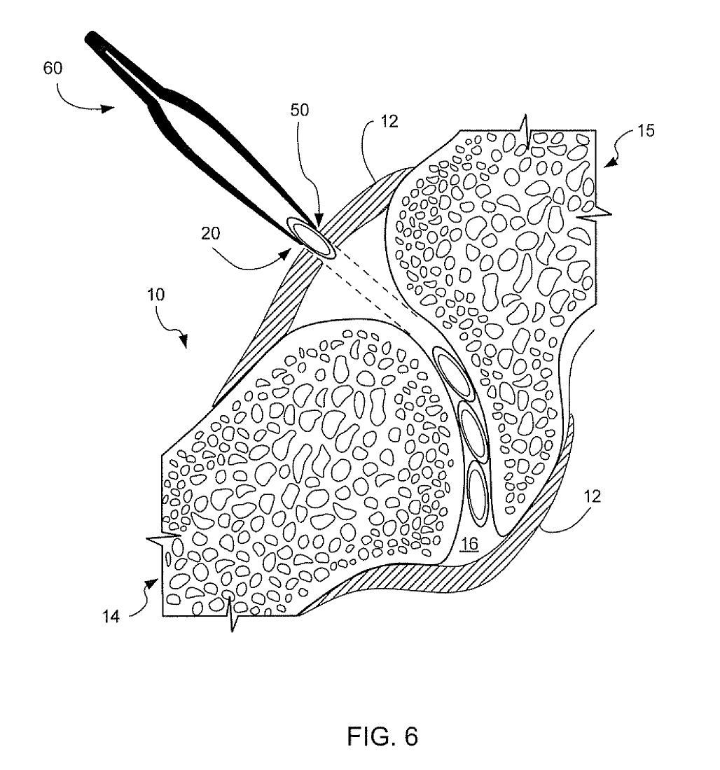

FIG. 6 is a graphical representation of a method of delivering an implantable device for joint repair, according to one embodiment of the invention. In this example, tweezers 60 is used to deliver implantable device 20 into cavity 16 of facet joint 10 through hole 50. As illustrated in FIG. 6, additional implantable device(s) may be placed in cavity 16 between bones 14 and 15 of facet joint 10. In the example shown in FIG. 6, tweezers 60 is used to deliver implantable device 20 containing inner core 26. In one embodiment, tweezers 60 may be used to deliver outer layer 22 of implantable device 20 first.

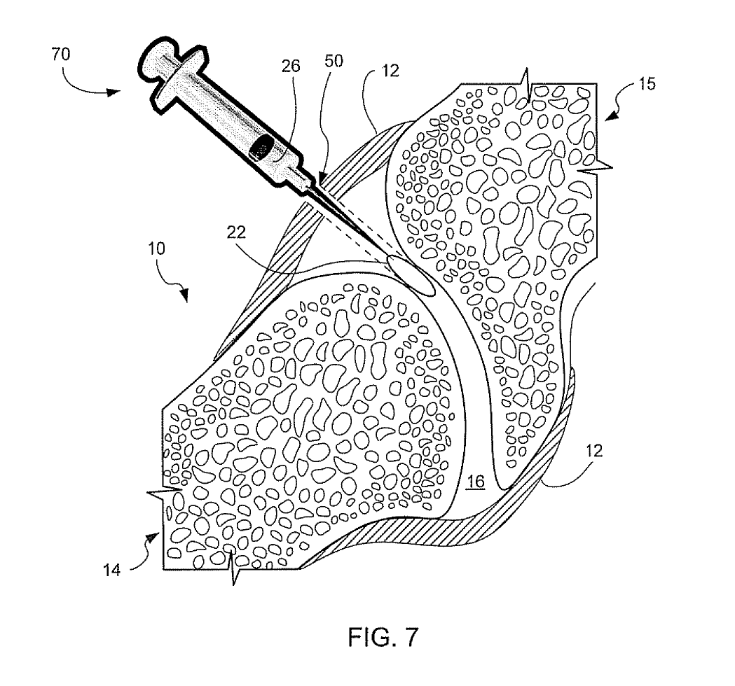

Inner core 26 can be pre-formed, in-situ curable, or injectable, in which case, outer layer 22 may be inserted in place and the material of inner core 26 is then injected separately. FIG. 7 is a graphical representation of a method of delivering an implantable device for joint repair, according to one embodiment of the invention. In this example, outer layer 22 of implantable device 20 is first placed in cavity 16 of facet joint 10 through hole 50. As exemplified in FIG. 7, the material of inner core 26 can then be injected through outer layer 22 using syringe 70 which has a sufficiently long needle allowing syringe 70 to reach and puncture outer layer 22.

As described herein, the material of inner core 26 is an expandable polymeric material (e.g., a hydrogel), which swells until its equilibrium water content is reached. In one embodiment, outer layer 22 and inner core 26 are made of different hydrogels. In one embodiment, outer layer 22 is at least one order of magnitude stronger than inner core 26. In one embodiment, after implantable device 20 is introduced into cavity 16, it swells as it rehydrates. In one embodiment, upon absorbing water within facet joint 10, implantable device 20 expands in volume, effectively stopping itself from exiting cavity 16 through hole 50. It should be appreciated that, according to one embodiment of the invention, one or more implantable devices can be inserted in an articulating joint through one or more incisions. As described above, such incision(s) or hole(s) can be optionally sealed after the implantable device(s) has/have been introduced into the joint. It should also be appreciated that the figures are not drawn to scale.

Other methods of delivery are also possible. For example, in one embodiment, a minimally invasive surgical device/system can be used to place the implantable device in the desired place. Examples of suitable minimally invasive surgical devices/systems may include, but not limited to, Abbott Spine's PathFinder®, Harmony™ Port System, etc. PathFinder® is a minimally invasive device used in lower back surgery requiring only two small incisions, potentially reducing surgical pain and allowing patients to go home sooner. The Harmony™ Port System, also available from Abbott Spine, supports a minimally invasive approach to surgery while maximizing accessibility and visualization. Other minimally invasive surgery systems, devices, and platforms may also be utilized in delivering embodiments of the implantable device disclosed herein. According to one embodiment of the invention, after introduction, the implantable device can be contained, held, or otherwise constrained in the intended space via various temporary and/or permanent arrangements as described above using a variety of options.

As described above, embodiments of the invention provide two distinct parts. The outer layer is bioresorbable and the inner core is bioactive. The outer layer can be porous (e.g., 35% solid or coverage) to allow the inner core material to bond with the bone over time (e.g., about 2 weeks to three months). Porosity can depend on various factors (e.g., thickness of the actual space between joints, thickness/composition of the outer layer material, volume of the inner core, ability of the inner core to bond with the bone, etc.).

The outer layer material can be a polymeric, strong material. By way of example and not limitation, the outer layer can be stronger as compared to the inner core material. For example, a difference in strength between the outer layer and the inner core material can be at least one order of magnitude.

In one embodiment, the inner core material of the implantable device is a class of biocompatible material called hydrogel, which is a jelly-like material and can self-cure with or without heat. Where applicable, heat can be generated from a variety of sources (e.g., human body, radiation, etc.)

There are several families of hydrogel known in the art, all of which can absorb water and swell in volume (e.g., a hydrated hydrogel may be three times larger than its dehydrated form). Hydrogel can be fully hydrated when introduced into the facet joint, or can be, for example, introduced as a swellable material (e.g., a dehydrated sheet) that attracts water and swells/rehydrates once introduced into the joint. Replication Medical, Inc. of New Brunswick, N.J., USA, is developing a spinal disc nucleus replacement device based on a two-phase, expandable hydrogel that has anisotropic expansion (swelling) properties that ideally suit it for the intervertebral disc space. The nucleus replacement device swells and contracts to fill the disc space; it swells when a person is “downloaded” at night, and compresses water out when the spine is loaded. In some embodiments of the invention disclosed herein, the hydrogel significantly expands only in a specified direction (e.g., X-Z) and not in other two directions (e.g., X-Y, Y-Z, etc.). According to the invention, the expansion direction of the hydrogel can be manipulated.

According to some embodiments of the invention, suitable exemplary outer layer materials include poly lactic acid (PLA), poly lactic-glycolic acid (PLGA), polyethylene glycol (PEG), bioglass, metal matrix composites such as magnesium-HA, poly (amino acid) hydrogel and its copolymers, etc. According to some embodiments of the invention, suitable exemplary inner core materials include hydrogels and their copolymers such as poly (vinyl alcohol) PVA, poly (vinyl pyrrolidone) PVP, silicone, polyacrylates (PA), poly (acrylonitrile) PAN, synthetic recombinant protein hydrogel, polyurethanes, polyethylene, polycarbonate and their co-polymers, etc. The lists of materials are not meant to be exclusive as other polymers can also be used in the practice of this invention.

Without modification, hydrogel is a very soft material and cannot survive the normal spine load. For this reason, hydrogel is generally not considered a load-bearing material. In embodiments of the invention, the polymeric outer layer material has physical cross-linking, which provides stronger mechanical strength than chemical cross-linking, allowing the implantable device to act as a load-bearing device, and which protects the liquid material of the inner core, allowing the inner core material to properly cure inside the human body. According to one embodiment of the invention, the polymeric outer layer can be made utilizing a physical cross-linking method comprising the following steps:
adding powders of a first polymer and a second polymer with distilled water in a container;
stirring the solution for half an hour;
heating the container containing the solution in an incubator at 80 degree C. for about 8 to 12 hours;
removing the container from the incubator;
determining whether any trace of the powders remains in the solution;
if so, stirring the solution again until no trace of the powders can be seen in the solution;
casting the solution in one or more molds of desired shape(s); and
performing repeated freeze-thaw cycles of 21 hours-3 hours.

In one embodiment, the inner core can be made in a similar way, with lower number of freeze-thaw cycles. This produces hydrogels with lower modulus. When cured, the inner core can act as a load-bearing material. The property of the load-bearing device can be optimized. As one of ordinary skill in the art can appreciate, embodiments of the invention disclosed herein can be made with any physically cross-linked hydrogels. Other physical cross-linking methods can also be used to manufacture the outer layer and the inner core. For example, in “Cellular VA Hydrogels Produced by Freeze/Thawing,” Journal of Applied Polymer Science 76 (2000) 2075-2079, which is incorporated herein by reference, Hassan et al. describe that cellular poly(vinyl alcohol) (PVA) hydrogels prepared by a freeze/thaw method showed overall enhanced swelling with increased mechanical strength over traditional hydrogels prepared by chemical or irradiative cross-linking techniques. As another example, in “Novel Crosslinking Methods to Design Hydrogels,” Advanced Drug Delivery Reviews 54 (2002) 13-36, which is incorporated herein by reference, Hennink et al. describe that unwanted reactions with bioactive substances present in the hydrogel can be avoided with the use of physically cross-linked hydrogels.

In addition to acting as a load-bearing device, the outer layer is flexible and, in conjunction with the inner core, allows the implantable device according to the present invention to act as a cushion. One additional advantage is that it also allows the implantable device according to the present invention to be attachable via the outer layer (e.g., screwed to site).

In some embodiments, the outer layer can be made of a pre-formed material, a pre-cast material, or an injection-molded polymeric material. In some embodiments, the outer layer may be made of a polymer or a composite material, including a variety of combinations of polymers, metals, and ceramics materials (e.g., polymer-metal, polymer-ceramic, polymer-metal-ceramic, polymer-ceramic-metal, etc.). Additional representative examples of suitable polymeric materials are described in U.S. Pat. Nos. 5,976,186, 6,264,695, 6,280,475, 6,443,988, and 6,595,998, each of which is incorporated herein by reference in their entirety.

In some embodiments, the polymeric materials (i.e., the inner core and/or the outer layer) can contain a variety of other additives, such as pharmaceutically-active compounds, analgesics, antibiotics, nutrients, building blocks for tissue generation, and so on. Additionally, in some embodiments, a lubricating composition may be introduced (e.g., additional synovial fluid, hyaluronic acid, etc.). Furthermore, in some embodiments, radiographic markers (e.g., one or more strips of a tantalum wire) may also be included. In some embodiment, an adhesion or fibrosis-inducing agent may be included to promote scarring and fixation of embodiments of the implantable device disclosed here into the surrounding bone. The addition of a fibrosis-inducing agent may enhance efficacy and longevity of the joint repair procedure in several ways. For example, inducing fibrous tissue around the implantable device can secure the implantable device in place for joint repair, allowing it to maintain the proper position in the joint. Exemplary fibrosing agents include talc, silk, wool, chitosan, polylysine, fibronectin, bleomycin, and CTGF, as well as analogues and derivatives thereof. Additional representative examples of suitable fibrosis-inducing agents and methods of incorporating fibrosis-inducing agents are described in U.S. Pat. No. 7,166,570, which is incorporated herein by reference in its entirety.

Embodiments of the invention provide a number of advantages, including but not limited to the following. The implantable device can provide a surface to enhance lubrication within the facet joint, which can reduce pain associated with degenerated facet joints. As one skilled in the can appreciate, embodiments of the invention are not limited for spine applications and can be applied to repair any joints in the body. Given the shape and simplicity of the design of the implantable device disclosed herein, the delivery can be done using minimally invasive techniques as described above with reference to FIGS. 5-7.

Both the outer layer and inner core of the implantable device can be made of relatively inexpensive materials that are compatible with the human body. The implantable device can be made relatively inexpensively due to its straightforward design and yet provide different desirable features at the same time. For example, the outer layer of the implantable device is flexible enough to allow the inner core to swell if desired and yet strong and stiff enough to hold the inner core material and maintain the separation of the articulating joints for a period of time. In this way, the implantable device advantageously serves as a cushion between the articulating joints, minimizing the friction between them, thereby reducing or eliminating the debilitating pain generated as a result of nerve fibers in contact. Moreover, the implantable device can advantageously serve as a load-bearing device, offloading some or all of the physiologic loads from the degenerative facet joints and alleviating pain associated therewith. As the need for spinal fusion/fixation procedures is eliminated by the insertion of implantable device(s), the intended natural function of articulating surfaces can be beneficially preserved and/or restored.

As one skilled in the art can appreciate, embodiments of the invention disclosed herein can be modified or otherwise implemented in many ways without departing from the spirit and scope of the invention. Accordingly, this description is to be construed as illustrative only and is for the purpose of teaching those skilled in the art the manner of carrying out the invention. It is to be understood that the forms of the invention herein shown and described are to be taken as exemplary embodiments. Equivalent elements or materials may be substituted for those illustrated and described herein. Moreover, certain features of the invention may be utilized independently of the use of other features, all as would be apparent to one skilled in the art after having the benefit of this description of the invention.

What is claimed is:

1. An implantable device for repairing an articulating joint, comprising:
an outer layer made of a first polymeric material; and
an inner core made of a second polymeric material;
where said first polymeric material is at least partially bioresorbable;
wherein said second polymeric material is bioactive and expandable; and
wherein said first polymeric material is at least one order of magnitude stronger than said second polymeric material, allowing said implantable device to act as a load-bearing device, a separator, and a cushion for said articulating joint.

2. The implantable device of claim 1, wherein said first polymeric material and said second polymeric material are two different hydrogels.

3. The implantable device of claim 1, wherein said second polymeric material is a hydrogel.

4. The implantable device of claim 1, wherein said outer layer and said inner core are made of two different polymeric materials.

5. The implantable device of claim 1, wherein size, shape, thickness, and dissolvance time of said outer layer are manipulatable.

6. The implantable device of claim 1, wherein said outer layer is configured to remain at least partially unabsorbed for at least 2 weeks after implantation.

7. The implantable device of claim 1, wherein said outer layer is porous.

8. The implantable device of claim 7, wherein porosity of said outer layer is manipulatable to affect bonding time of said inner core.

9. The implantable device of claim 1, wherein said outer layer has a plurality of protruding elements for anchoring.

10. The implantable device of claim 1, wherein bioactivity, bonding ability, and at least one mechanical property of said inner core are manipulatable.

11. The implantable device of claim 1, wherein said inner core is swellable in human body.

12. The implantable device of claim 10, wherein swellability of said inner core is enhanced with a fluid.

13. The implantable device of claim 1, wherein said inner core is self-curable in a human body.

14. The implantable device of claim 1, wherein said implantable device is attachable to a human body.

15. A method for repairing a facet joint, comprising:
making a minimally invasive incision into said facet joint, wherein said minimally invasive incision is of sufficient size to permit insertion of an implantable device into a cavity within said facet joint;
introducing at least an outer layer of said implantable device into said cavity of said facet joint via said minimally invasive incision, wherein said implantable device further comprises an inner core, wherein said outer layer and said inner core are made of two different polymeric materials, wherein said outer layer is at least partially bioresorbable, wherein said inner core is bioactive, wherein said outer layer is at least one order of magnitude stronger than said inner core, and wherein said implantable device is capable of acting as a load-bearing device, a separator, and a cushion for said facet joint.

16. The method of claim 15, further comprising:
distracting said facet joint prior to said introducing step.

17. The method of claim 15, further comprising:
injecting said inner core into said outer layer of said implantable device.

18. The method of claim 15, further comprising:
closing said minimally invasive incision.

19. The method of claim 15, further comprising:
holding said implantable device in a space between articulating surfaces of said facet joint via a temporary arrangement, a permanent arrangement, or a combination thereof.

20. An implantable device for repairing a facet joint, comprising:
an outer layer; and
an inner core;
wherein said outer layer is at least partially bioresorbable;
wherein said inner core is bioactive;
wherein said outer layer is stiffer than said inner core; and
wherein said implantable device acts as a load-bearing device, a separator, and a cushion for said facet joint.

21. The implantable device of claim 20, wherein said outer layer is porous.

22. The implantable device of claim 20, herein said inner core is made of a polymeric material capable of bonding with said facet joint.

23. The implantable device of claim 22, wherein bioresorbability of said outer layer is manipulatable to affect said bonding with said facet joint.

24. The implantable device of claim 22, wherein bioactivity of said inner core is manipulatable to affect said bonding with said facet joint.

25. The implantable device of claim 20, wherein said outer layer has a plurality of protruding elements.

26. An implantable device for repairing a facet joint, comprising:
an outer layer and an inner core;
wherein said outer layer is porous and not bioresorbable;
wherein said inner core is bioactive; and
wherein said implantable device acts as a load-bearing device, a separator, and a cushion for said facet joint.

27. The implantable device of claim 26, wherein said bioactive inner core is made of a polymeric material capable of bonding with said facet joint.

28. The implantable device of claim 27, wherein porosity of said porous outer layer is manipulatable to affect said bonding with said facet joint.

29. The implantable device of claim 27, wherein bioactivity of said inner core is manipulatable to affect said bonding with said facet joint.

30. The implantable device of claim 23, wherein said outer layer has a plurality of protruding elements.
